(19) United States
(12) Patent Application Publication
(10) Pub. No.: US 2014/0158124 A1
(43) Pub. Date: Jun. 12, 2014

(54) METHOD AND SYSTEM FOR THE DELIVERY OF CARBON DIOXIDE TO A PATIENT

(71) Applicants: Erwan L'her, Brest (FR); François Lellouche, Lac Beauport (CA); Frédéric Series, Quebec (CA)

(72) Inventors: Erwan L'her, Brest (FR); François Lellouche, Lac Beauport (CA); Frédéric Series, Quebec (CA)

(73) Assignees: UNIVERSITÉ DE BRETAGNE OCCIDENTALE, Brest (FR); UNIVERSITÉ LAVAL, Québec, QC (CA)

(21) Appl. No.: 13/979,525

(22) PCT Filed: Jan. 12, 2012
(86) PCT No.: PCT/IB12/50163

§ 371 Date: Feb. 18, 2014

Related U.S. Application Data
(60) Provisional application No. 61/432,371, filed on Jan. 13, 2011.

(51) Int. Cl. A61M 16/00; A61B 5/00; A61M 16/20; A61B 5/113; A61B 5/0205; A61B 7/00; A61M 16/12; A61B 5/145

(52) U.S. Cl. CPC A61M 16/0051; A61M 16/12; A61B 5/4848; A61B 5/4839; A61B 5/14542; A61B 5/113; A61B 5/4812; A61B 5/4818; A61B 5/0205; A61B 7/003; A61M 16/20; A61B 5/0816
USPC 128/203.14

(57) ABSTRACT

A method and system for delivering a gas containing carbon dioxide to a patient are described. The method comprises measuring a physiological parameter of breathing stability in the patient; determining an optimal gas delivery parameter based on the physiological parameter of breathing stability; and delivering the gas to the patient in accordance with the optimal gas delivery parameter.

### CROSS-REFERENCE TO RELATED APPLICATIONS

This application claims priority under 35USC§119(e) of U.S. provisional patent application 61/432,371 filed Jan. 13, 2011 and is related to U.S. patent application Ser. No. 12/837,259 filed on Jul. 15, 2010 and published on Mar. 24, 2011 as US 2011/0067697, the specifications of which are hereby incorporated by reference.

### TECHNICAL FIELD

The invention relates to a device and method for the delivery of gas containing carbon dioxide (CO₂) to a patient and more particularly to a controlled delivery based on the detection of a breathing disorder.

### BACKGROUND OF THE ART

Sleep disordered breathing (SDB) is characterized by irregular breathing both in rate and depth (amplitude). SDB can include periodic hypopnea (overly shallow breathing or an abnormally low respiratory rate) and periodic apnea (no breathing). It is established that SDB has two main causes: 1) obstructive abnormalities, which are associated with an obstruction of the pharyngeal airway and 2) central sleep disorders, which stem from a failure of the sleeping brain to generate regular rhythmic neural signals needed by the respiratory muscles.

Obstructive abnormalities can usually be treated using positive airway pressure (PAP) therapy, where a breathing gas is introduced in the airways of the patient at a pressure slightly higher than the atmospheric pressure. However, central sleep disorders are not treated effectively with PAP, even with the administration of oxygen-enriched breathing gases (oxygen therapy).

Disturbed sleep usually results in chronic fatigue, and impairs the patient's daytime cognitive functions and quality of life. SDB is frequently observed in patients with heart failure. For these patients, central sleep apnea is a serious condition that is believed to aggravate cardiac arrhythmia and to increase the occurrence of strokes and myocardial infarctions. Unfortunately, there exist no approved methods for the treatment of central sleep apnea.

The most well-known central sleep disorder is the Cheyne-Stokes respiration (CSR) where a patient experiences a succession of hyper- and hypoventilation periods. This type of disorder is mainly experienced late at night, during nights where obstructive apnea/hypopnea episodes were observed in the early hours of sleep. CSR can also be observed at any time of the night and even during wake time in advanced forms of heart failure. The prevalence of CSR in the population with congestive heart failure is estimated at between 15 and 35%.

The central respiratory function is a complex system that comprises multiple feedback mechanisms based on chemical receptors sensing carbon dioxide (CO₂), oxygen (O₂) and blood acidity (pH). When the feedback signals are not sufficiently intense, the central rhythmic neural signals to the respiratory muscles are perturbed or can stop completely. Hyperventilation associated with unstable breathing also contributes to lower the blood concentration of CO₂.

It has been shown that increasing the concentration of CO₂ in the breathing air has a stabilizing effect on patients with CSR, because of the increased CO₂ feedback signal. However, no practical methods for administering CO₂ to a patient are commercially available.

A prior art method of administering CO₂ relies on the accepted PAP technique. PAP requires leak-proof masks that are uncomfortable because they need to be secured tightly over the patient's face. PAP gases with low humidity content also contribute to the drying of the respiratory passageways and the patient's discomfort. One should note that the administration of a continuous flow of CO₂, such as is proposed in this prior art method, is a significant medical expense due to the large quantities of gas used.

An alternate prior art method utilizes a dead space in an external breathing apparatus as a simple way to increase the fractional concentration of inspired CO₂ (FICO2). This method has the disadvantages of requiring a leak-proof mask and demanding an increased respiratory effort to move the gases in the external breathing circuit.

### SUMMARY

According to one broad aspect of the present invention, there is provided a method for delivering a gas containing carbon dioxide to a patient. The method comprises measuring a physiological parameter of breathing stability in the patient; determining an optimal gas delivery parameter based on the physiological parameter of breathing stability; and delivering the gas to the patient in accordance with the optimal gas delivery parameter.

In one embodiment, the gas containing carbon dioxide is a mixture of gases including carbon dioxide.

In one embodiment, the method further comprises repeating the step of measuring the physiological parameter of breathing stability in the patient, after the delivering the gas, to determine an effect of the delivering on the physiological parameter.

In one embodiment, the method further comprises repeating the steps of determining the optimal gas delivery parameter and delivering the gas to adjust the delivering consequently to the effect.

In one embodiment, the optimal gas delivery parameter is selected from the group consisting of a fraction of carbon dioxide in the gas and a flow rate of the gas during the delivering.

In one embodiment, the method further comprises issuing an alarm if the physiological parameter is measured to be outside of a predetermined threshold.

In one embodiment, the physiological parameter is the breathing pattern for the patient, the breathing pattern including at least the respiratory amplitude.

In one embodiment, the physiological parameter is analyzed to obtain a breathing pattern index for the patient and the determining the gas delivery parameter is carried out using the breathing pattern index.

In one embodiment, the physiological parameter further includes at least one parameter selected from the group consisting of arterial hemoglobin oxygen saturation, respiratory rate, respiratory amplitude, chest movement pattern, end tidal CO₂ (ETCO₂) level, Rapid Eye Movement (REM) pattern, rate of apnea, rate of hypopnea, rate of desaturation, respiratory rate variability, heart rate variability, heart rate synchrony and snoring noise level.

According to another broad aspect of the present invention, there is provided a system for delivering a gas containing carbon dioxide to a patient. The system comprises a physiological sensor for measuring a physiological parameter of breathing stability in the patient; a controller receiving the physiological parameter from the physiological sensor for determining an optimal gas delivery parameter based on the physiological parameter of breathing stability; and a gas delivery sub-system having a gas source and a gas delivery controller for delivering the gas to the patient in accordance with the optimal gas delivery parameter received from the controller.

In one embodiment, the gas containing carbon dioxide is a mixture of gases including carbon dioxide.

In one embodiment, the optimal gas delivery parameter is selected from the group consisting of a fraction of carbon dioxide in the gas and a flow rate of the gas during the delivering and wherein the gas delivery controller uses the gas delivery parameter to deliver the gas from the source.

In one embodiment, the system further comprises an alarm sub-system including an alarm emitter and an alarm controller, the alarm controller having a predetermined threshold, the alarm controller receiving the physiological parameter from the controller and controlling the alarm emitter to issue an alarm if the physiological parameter is measured to be outside of the predetermined threshold.

In one embodiment, the physiological parameter is the breathing pattern for the patient, the breathing pattern including at least the respiratory amplitude.

In one embodiment, the system further comprises a breathing pattern index calculator for analyzing the physiological parameter to obtain a breathing pattern index for the patient and wherein the controller uses the breathing pattern index to determine the gas delivery parameter.

In one embodiment, the physiological parameter further includes at least one parameter selected from the group consisting of arterial hemoglobin oxygen saturation, respiratory rate, respiratory amplitude, chest movement pattern, end tidal CO₂ (ETCO₂) level, Rapid Eye Movement (REM) pattern, rate of apnea, rate of hypopnea, rate of desaturation, respiratory rate variability, heart rate variability, heart rate synchrony and snoring noise level.

In one embodiment, the system further comprises an analysis module for analyzing the measured physiological parameter and determined gas delivery parameter to detect a trend for the patient.

### BRIEF DESCRIPTION OF THE DRAWINGS

Having thus generally described the nature of the invention, reference will now be made to the accompanying drawings, showing by way of illustration a example embodiment thereof and in which

FIG. 1 is a schematic illustration of an example embodiment;

FIG. 2 is a functional block diagram of the main components of an example embodiment;

FIG. 3 is a graph of an example breathing pattern plotted against the time;

FIG. 4 is a graph of an example expired CO₂ concentration plotted against the time;

FIG. 5 is a flowchart illustrating the main steps of an example method for delivering the CO₂ to a patient with the example system shown in FIG. 1;

FIG. 6 includes FIG. 6A and FIG. 6B, wherein FIG. 6A is a graph of an example breathing pattern with some low amplitude respirations plotted against the time and FIG. 6B is a graph of an example delivery of CO₂ in response to the breathing pattern shown in FIG. 6A; and

FIG. 7 includes FIG. 7A and FIG. 7B, wherein FIG. 7A is a graph of an example expired CO₂ concentration with some low end tidal CO₂ (ETCO₂) values plotted against the time and FIG. 7B is a graph of an example delivery of CO₂ in response to the respiratory pressure shown in FIG. 7A.

It will be noted that throughout the appended drawings, like features are identified by like reference numerals.

### DETAILED DESCRIPTION

The present invention proposes an adaptive system and method where CO₂ is delivered based on the patient physiological data with the aim to stabilize, or at least improve, the breathing pattern. The physiological parameter detected is therefore indicative, in some respect, of breathing stability.

A closed control loop is used to deliver CO₂ intermittently in response to respiratory abnormalities or patterns, thereby helping to reduce central apnea and hypopnea. The quantity of CO₂ used in the proposed method and system is reduced with respect to existing systems which deliver CO₂ since the CO₂ is administered according to delivery parameters (flow rate, time and duration) determined using measured physiological data. In most cases the administration of CO₂ will be intermittent, thus greatly reducing the amount of delivered CO₂ compared with a continuous delivery.

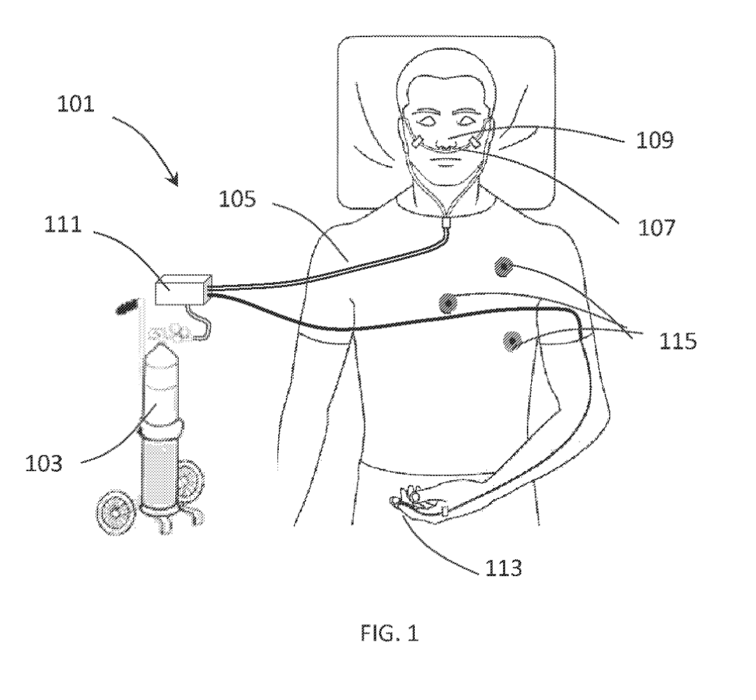

FIG. 1 is a schematic illustration of an example system 101 used to administer gaseous CO₂ from a CO₂ source 103 to a patient 105 by means of a nasal cannula 107 affixed to the patient's nose 109. The quantity of CO₂ delivered to the patient 105 from the source 103 is controlled using the integrated system 111.

Sensors are used to provide physiological signals that can be utilized by the integrated system 111 to change the amount of CO₂ administered to the patient 105. At least one breathing pattern sensor 115, for example an accelerometer, detects the breathing pattern (depth (amplitude) of breath, rate, presence or absence of breath, etc.) of the patient and sends its signal to the integrated system 111. The breathing pattern could only detect amplitude of breath but typically detects both amplitude and rate. The integrated system 111 uses this physiological signal to adjust the delivery of CO₂.

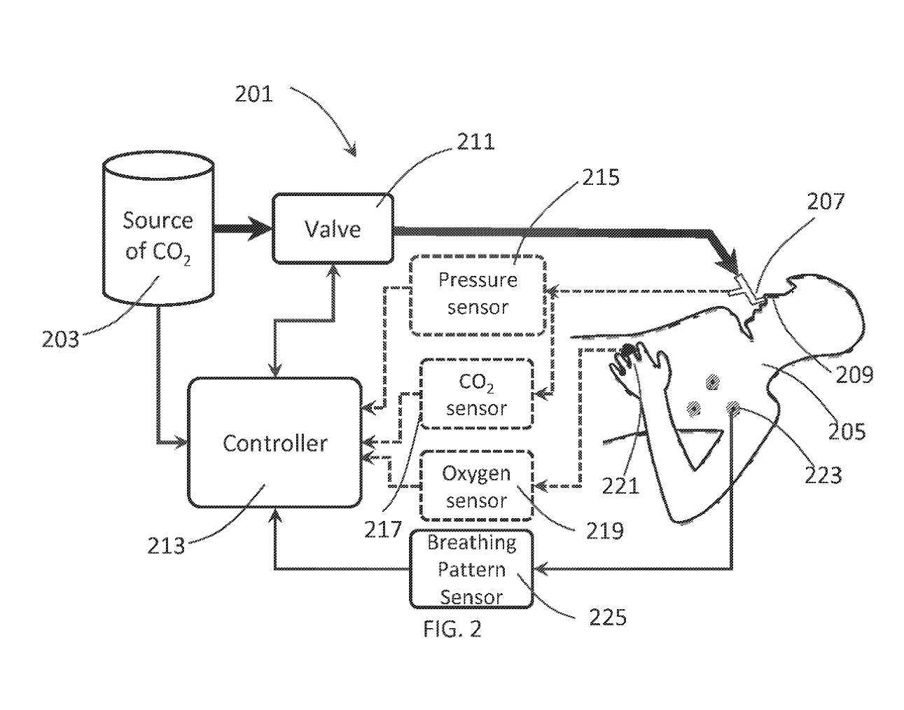

The nasal cannula 107 can optionally include a pressure sensor and can also optionally include an end tidal CO₂ (EtCO₂) sensor as will be depicted in FIG. 2. A blood oxygen sensor (oxymeter or SpO₂ sensor) 113 can also optionally be used with the system. The physiological signals acquired by the optional pressure sensor, EtCO₂ sensor and oxymeter can also be used by the integrated system 111 to adjust the delivery of CO₂.

FIG. 2 is a functional illustration of an example system 201 used to administer gaseous CO₂ from a source 203 to a patient 205 by means of a nasal cannula 207 affixed to the patient's nose 209. The quantity of CO₂ delivered to the patient 205 from the source 203 is controlled using a motorized proportional valve 211 commanded by a controller 213.

The motorized proportional valve 211 has an actuator (not shown) which allows a displaceable portion of the valve 211 to be moved between a closed position and an open position to allow the flow of CO₂ to be sent to the patient 205 from the source 203. As will be readily understood, a partial opening is also possible to control the flow of CO₂. The valve 211 may or may not provide feedback information regarding its degree of opening to the controller, which may differ from the commanded value.

The controller 213 receives physiological signals from the patient and calculates the appropriate command for the valve 211. In the example embodiment, the physiological signals can include the breathing pattern obtained from the breathing pattern sensor 225, for example accelerometer 223, the breathing amplitude and rate derived from pressure sensor 215, the expired CO₂ concentration derived from CO₂ sensor 217, as well as the arterial hemoglobin (blood) oxygen saturation measured by pulse oximetry (SpO₂) using O₂ sensor (oxymeter) 219 and the derived heart rate. In one example embodiment, only the expired CO₂ concentration derived from CO₂ sensor 217 is used by the controller 213 as a physiological signal. In another example embodiment, only the breathing pattern obtained from the breathing pattern sensor 225 is used by the controller 213 as a physiological signal.

Examples of physiological signals that can be tracked to evaluate the quality of sleep of the patient after delivery of CO₂ include the Rapid Eye Movement (REM) pattern, breathing pattern (respiratory flow, respiratory pressure, rate of apnea, rate of hypopnea, rate of desaturation, respiratory rate variability), heart rate variability, heart rate synchrony, movement of patient, electromyogram of muscles involved in breathing (for example from nasal muscles to intercostal muscles, diaphragm of sternocleido mastoids, etc.), detection of thoracic movements by plethysmography or other suitable method, the patient's temperature and the patient's snoring noise level. A quality of sleep parameter can be obtained using these physiological signals and can be used by the controller 213 to adjust the command for the valve 211.

The CO₂ source 203 is usable for providing a gas including CO₂ to the patient 205. In some embodiments of the invention, the gas source 203 is a CO₂ source providing a pre-determined concentration of CO₂ to the patient. This pre-determined concentration can be set to any useful concentration, for example a 100% concentration corresponds to pure CO₂. In these embodiments, the controller 213 is usable for controlling a gas flow rate of the gases source 203. In other embodiments of the invention, the gases source 203 provides a mixture of air and CO₂. In these embodiments, the controller 213 is usable for adjusting a fraction of CO₂ in the gas and the gas flow rate of the gas source 203. In some embodiments, the source of CO₂ could be the expired gas from the patient. In yet other embodiments of the invention, any other suitable gas source 203 is used. The mixture of gas delivered to the patient may or may not include oxygen.

As will be readily understood, any suitable gas delivery apparatus including a facial mask, a venturi mask and eyeglasses provided with gas delivery tubes can be used instead of the nasal cannula 207.

The present invention provides an improved level of comfort for the patient. If the gas delivery apparatus is a mask, it does not have to be completely leak-proof. The comfort may be even further improved by having the patient wear a simple nasal cannula. Because the system has a retroaction via the physiological signals from sensors 215, 217, 219 and 225, the system is able to compensate for small leaks.

In the example shown, the breathing pattern sensor 225 is used to monitor the respiratory cycles and determine phases of hypo- and hyperventilation and the respiratory amplitude. Accelerometer-based respiratory monitoring is based on the observation of small rotations at the chest wall due to breathing. MEMS accelerometers worn on the torso can measure inclination changes due to breathing, from which a respiratory amplitude and/or rate can be obtained. Tri-axial accelerometer data can track the axis of rotation and obtain angular rates of breathing motion. Other types of breathing pattern sensors can include an infra-red reflector monitored by a camera, a spirometer, a belt connected to a bellows or an inductive belt.

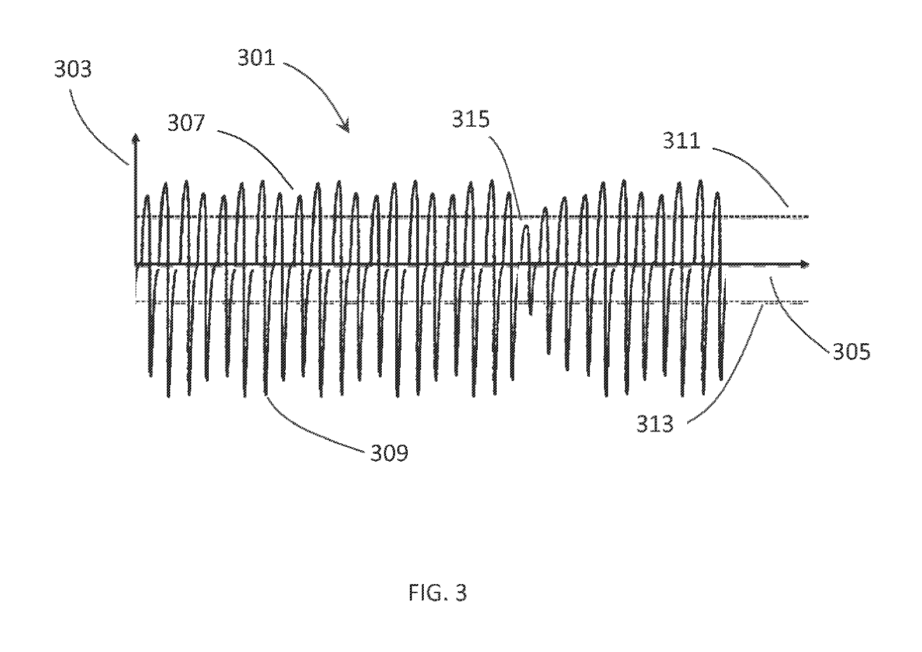

FIG. 3 is a graph 301 of the breathing pattern 303 obtained with the breathing pattern sensor 225, plotted against the time 305. A normal breathing pattern measured via the movements of the chest of the patient is composed of positive peaks 307 measured during inspiration when the chest stretches and negative peaks 309 measured during expiration when the chest deflates. As will be readily understood, a correlation of the measured chest displacements with the breath volumes of each patient will be necessary. The normal respiratory amplitudes during the expiratory and inspiratory phases vary according to the physical condition, level of physical effort and health condition of each person. It is possible to establish an acceptable inspiratory threshold 311 and expiratory threshold 313 for each person, for example by analyzing the breathing pattern during wake time. Using these respiratory thresholds, it is possible to classify normal or abnormal respiration. For example, the maximum value of the inspiration 315 did not reach inspiratory threshold 311, so the inspiration 315 is considered abnormal. Hypo- and hyperventilation are defined by the occurrence of abnormal respiration for a certain number of respirations or a certain period of time. These thresholds are then optionally used by the controller 213 to adjust the delivery of CO₂.

FIG. 3 could also represent a graph of the respiratory pressure 303 obtained with the respiratory pressure sensor 215 and plotted against the time since both sensors will capture a volume reading. The inspiration as detected with the pressure sensor 215 will yield a negative peak and the expiration will yield a positive peak.

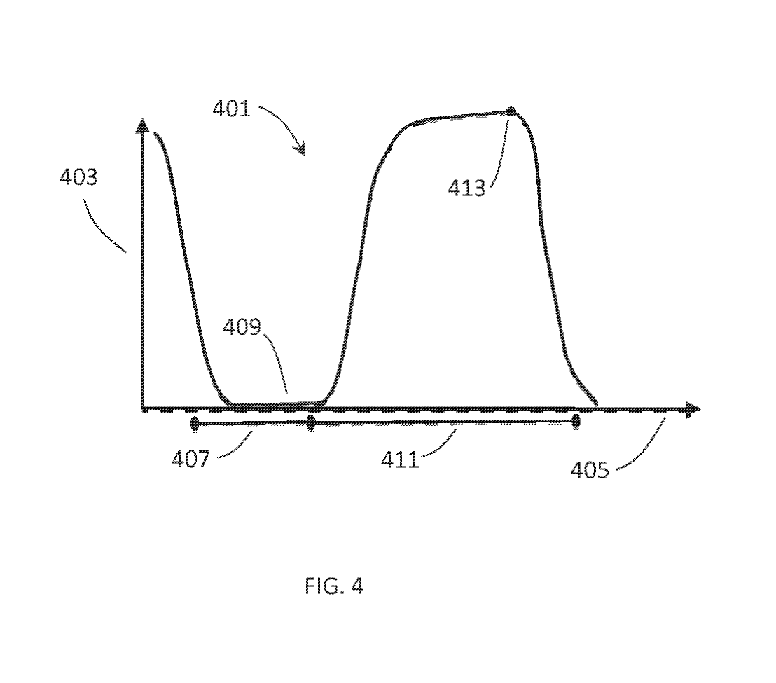

When the expired CO₂ concentration from the sensor 217 is used by the controller 213, a potential issue arises depending on the location of the CO₂ sensor. The sensor could sample not only the expired gases, but also the inspired gases. The presence of CO₂ in the inspiratory phase may result in potential measurement errors of the expired CO₂ parameter by the CO₂ sensor 217. FIG. 4 is a graph 401 of the CO₂ concentration 403 obtained with CO₂ sensor 217, plotted against the time 405. During the inspiratory phase 407, the CO₂ concentration drops to the value of the inspired air 409. During the expiratory phase 411, the CO₂ concentration increases to approximately 5%. The maximum value 413 reached at the end of the expiratory phase 411, is called the end tidal CO₂ (ETCO₂) concentration.

To reduce an impact of the potential issue of contamination of the expired CO₂ concentration measurement by inspired gases, the following algorithm can be used. Individual expiratory phases are identified and located in the CO₂ concentration versus time waveform by finding the places where the average over a typical expiratory period is maximized. Once the expiratory phases are located, the maxima of the measured values over each expiratory phase are extracted. These values correspond to the end tidal CO₂ concentrations and are free from inspired air contamination. These values are then optionally used by the controller 213 to adjust the delivery of CO₂

In another embodiment, the respiratory pressure sensor 215 can also be used in addition to the CO₂ concentration sensor 217 to determine or to improve the determination of when the inspiration and expiration phases begin and end, in order to reject data acquired during the inspiratory phase.

When the measurement of the blood oxygen saturation obtained using sensor 219 is used as a physiological signal, sensor 219 can take on different forms. In the example shown in FIG. 2, the blood oxygen saturation is obtained via a finger probe 221. In other embodiments, the blood oxygen saturation could be obtained via different means, such as using a toe probe or by placing an oximetry probe on another vascularized location on the body.

The controller 213 calculates the command to the proportional valve 211 as much as possible in real time in order to stabilize the condition of the patient shortly after a breathing anomaly or breathing pattern is detected by the controller based on the physiological data.

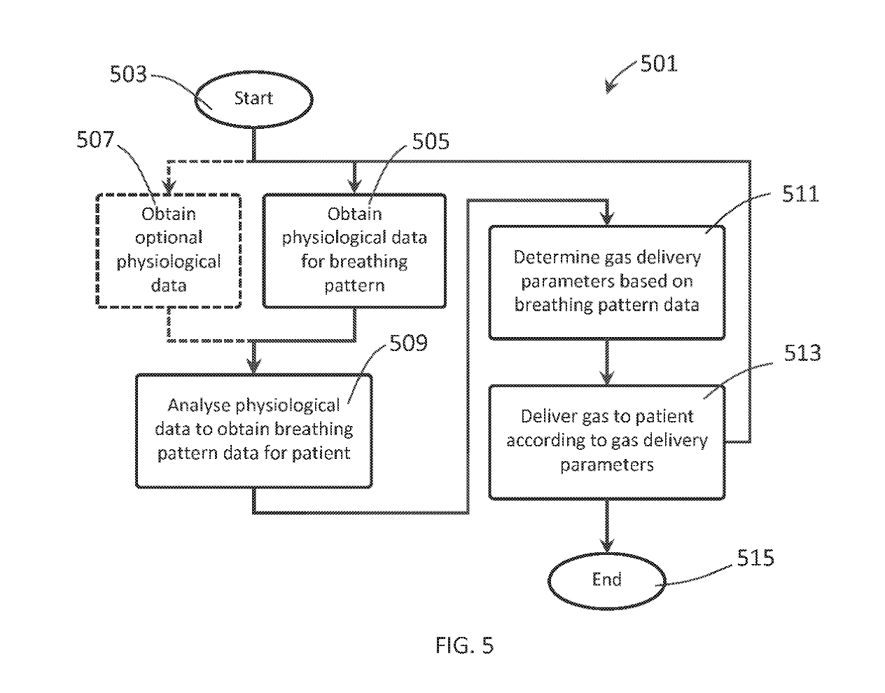

FIG. 5 is a flowchart illustrating an example method 501 for delivering the CO₂ to a patient 205. FIG. 5 will be described herein in relation with the system described in FIG. 2. After the system is powered up and initialized 503, the controller 213 reads at steps 505 and 507, the available physiological parameters, obtained with sensors 215, 217, 219 and 225. Next the controller 213 analyses 509 the available physiological parameters and derives a breathing pattern index. A breathing pattern index of 100% indicates normal breathing while a breathing pattern index of 0% indicates a completely disrupted breathing pattern. The breathing pattern index is automatically determined by the controller 213 based on the variations of the detected signals compared to the thresholds. These thresholds may have been determined for example during wake time or derived from studies and then provided to the controller during a set-up procedure.

The controller 213 also calculates 511 the amount of CO₂ to administer to the patient based on the available physiological data and breathing pattern index. The valve 211 is commanded 513 to the appropriate level allowing the CO₂ to be administered to the patient 205 as long as the breathing pattern is considered to be disordered. The steps in the method 501 are iterated continuously, for example several times per minutes, until the system is turned off 515, either by a trained person or by a system internal alarm.

The valve command is calculated using, for example, numerical servo computations based on the current values of the physiological signals as well as previous values measured in the preceding minutes. The function of the controller 213 can be implemented using a personal computer, but in the example embodiment, it is embedded in compact dedicated electronics composed of one or several micro-controllers, one or several digital signal processors (DSP), one or several field-programmable gate arrays (FPGA) or a combination of two or three of these types of electronic devices.

At step 511, the gas delivery parameters can be obtained using a proportional-integral-differential (PID) controller. Gas delivery parameters are determined in order to maintain one or several of the measured physiological parameters within a predetermined interval or as close as possible to a target value. In an embodiment of the invention, the breathing amplitude is derived from the physiological data obtained. A target value of, for example, more than 95% of the expiration amplitudes are larger than the expiratory threshold is selected. This target value can be adjusted according to the patient 205 in accordance with conventional criteria.

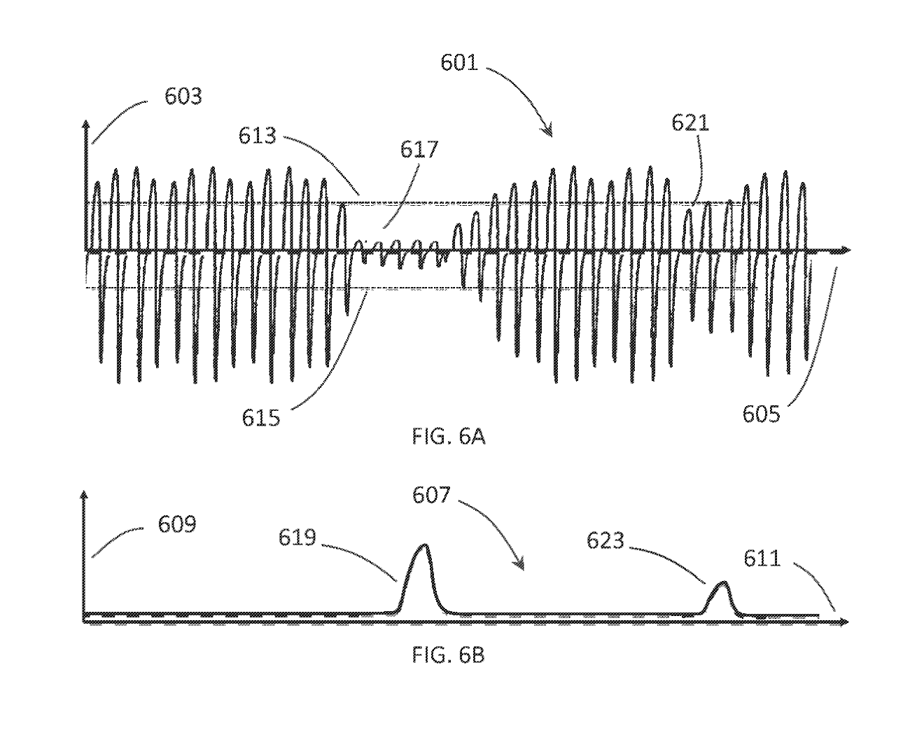

FIG. 6A is a graph 601 showing the breathing pattern 603 obtained with the breathing pattern sensor 225, plotted against the time 605. FIG. 6B is a graph 607 showing the amount of CO₂ 609 delivered by the controller 213, plotted against the time 611. The time scales 605 and 611 are the same. The inspiratory threshold 613 and expiratory threshold 615 are predetermined for each person. When the respiratory amplitudes are measured 617 to be lower than the thresholds for a certain period of time, the controller 213 can command the valve 211 to release a certain amount of CO₂ 619. When the respiratory amplitude returns to acceptable levels, the amount of CO₂ delivered can be nil. If a smaller deviation from the threshold is measured 621, a smaller amount of CO₂ 623 can be administered by the system by controlling the valve 211.

In another example embodiment of the invention, the measured physiological parameter is indicative of the expired CO₂ concentration in the patient and a target value of, for example, 40 mmHg is selected. This target value can be entered as a fixed parameter, adjusted according to the patient 205 in accordance with conventional criteria, including from data measured in a sleep evaluation laboratory or can be determined automatically by the controller 213 based on the acquired physiological data.

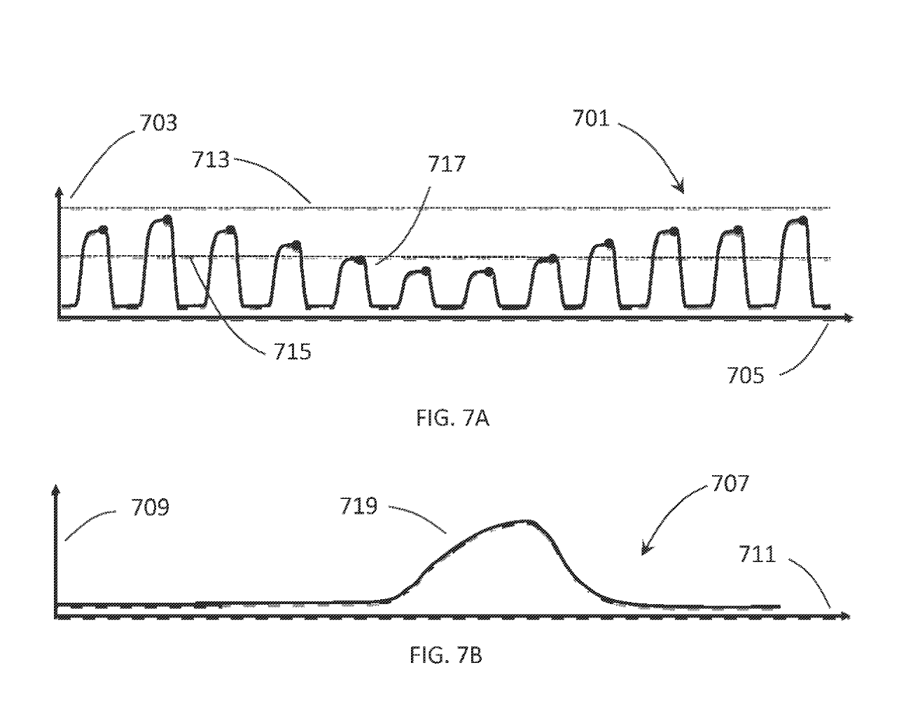

FIG. 7A is a graph 701 showing the expired CO₂ concentration 703 obtained with the CO₂ sensor 217, plotted against the time 705. FIG. 7B is a graph 707 showing the amount of CO₂ 709 administered by the controller 213, plotted against the time 711. The time scales 705 and 711 are the same. The expired CO₂ concentration is considered normal when it is lower than the upper limit 713 and higher than the lower limit 715. These limits are determined in accordance with conventional criteria, including from data measured in a sleep evaluation laboratory, as fixed parameters or adjusted automatically by the controller 213 based on the acquired physiological data. When the expired CO₂ concentration is measured 717 to be lower than the lower limit for a certain period of time, the controller 213 can command the valve 211 to release a certain amount of CO₂ 719. When the expired CO₂ concentration increases above the lower limit, the quantity of CO₂ delivered can be nil. When the expired CO₂ concentration is measured to be higher than the higher limit for a certain period of time, the controller 213 can trigger an alarm.

In yet another example embodiment of the invention, the measured physiological parameter is the respiratory rate of the patient and a target value of, for example, less than 30/min is selected. This target value can be adjusted according to the patient 205 in accordance with conventional criteria.

In yet another example embodiment of the invention, the breathing pattern index is derived from the physiological data obtained. A target value of, for example, 90% breathing pattern index is selected. This target value can be adjusted according to the patient 205 in accordance with conventional criteria.

At step 513, the valve 211 is operated so that the gas is administered to the patient in accordance with the optimal gas delivery parameters determined at step 511. This is typically performed by regulating the gas flow from source 203 with valve 211. Alternatively, a combination of proportional valves and on/off valves can be used to control the gas flow.

Safety mechanisms to limit the flow rate of administered CO₂ can be implemented. This can be done with a passive hardware flow limiter or with an active control approach using a flowmeter and a motorized limiter or safety valve.

The controller 213 determines the proper time of administration and amount of CO₂. For maximum efficiency, the administration of CO₂ would normally occur when the respiratory amplitude (quantity of air intake) is lower and would normally stop when it is returned to normal as illustrated in FIG. 6A and FIG. 6B. A dynamic and intermittent administration of CO₂ immediately proceeding and following hyperventilation is proposed.

In some embodiments of the invention, optional alarms can be issued if some of the physiological parameters are measured or calculated to be outside of predetermined intervals. Measured or calculated physiological parameters that may lead to the issuance of an alarm include, for example, respiratory amplitude and rate, expired CO₂ level, breathing pattern index, blood oxygen saturation, heart rate and temperature of the patient.

Examples of alarms that can be issued by an embodiment of the controller 213 are as follows: High End tidal CO₂ level (if this sensor is used), low SpO₂ level (if this sensor is used) or respiratory pressure (if this sensor is used) not available indicating that the nasal cannula is not in place should lead to an alarm.

Other examples of alarms that can be issued by an embodiment of the controller 213 are provided in the following list:

If the blood oxygen saturation is less than or equal to 85% for more than 3 seconds, a message indicating that connections of the blood oxygen saturation sensor 221 should be checked is issued and the method 201 steps back to step 203;

If the blood oxygen saturation is unmeasurable, a message indicating that connections of the blood oxygen saturation sensor 221 should be checked is issued and the desired CO₂ flow rate is set as a minimal safe flow rate, or as the last determined CO₂ flow rate;

If the expired CO₂ concentration is unmeasurable, a message indicating that connections of the CO₂ sensor 215 should be checked is issued;

If the expired CO₂ concentration is larger than or equal to 45 mmHg or has increased by more than 10 mmHg over the preceding hour, a message indicating the patient 205 should be closely monitored and that another CO₂ delivery technique may be preferable is issued;

If the expired CO₂ concentration is larger than or equal to 55 mmHg or has increased by more than 20 mmHg over the preceding hour, a message indicating that another CO₂ delivery technique may be preferable is issued.

The analysis of the data collected during periods where the CO₂ delivery system is used, for example during one night, can be performed automatically to provide a summary report of events after each operation period. It can include the amount of CO₂ delivered, a graph of the expired CO₂ concentration vs time, the number of apnea and hypopnea events, a graph of the respiratory amplitude and rate vs time, a graph of the breathing pattern index vs time, the number of desaturations (SpO₂<90%) and deep desaturations (SpO₂<80%), a graph of the blood oxygen saturation (SpO₂) level vs time, etc. Trends in the evolution of these parameters can also be made available for monitoring longitudinal changes in these patients.

The method allows monitoring by telemetry in the patients.

The proposed method and system can be used for the administration of CO₂ for a very wide range of clinical settings, in hospital setting for initial adaptations (sleep laboratory or respiratory ward) or at home from pre-hospital care to intra-hospital care (emergency department, intensive care units, respiratory/cardiology/internal medicine wards, rehabilitation units, post-anesthesia recovering rooms, for example). It can be used in portable settings, such as in ambulance vehicles, in camp sites during mountain climbing expeditions and the like. It can be used by patients at home for chronic respiratory and cardiac insufficiency and any cause resulting in breathing disorders. It can be used for adults or pediatric patients.

The proposed method 201 is typically performed without mechanically assisted ventilation of the patient 205. However, in alternative embodiments of the invention, such mechanical ventilation is used. In case of breathing disorders in mechanically ventilated patients, this technique and algorithm may be used to stabilize or help improve the breathing pattern and the resulting sleep quality.

While illustrated in the block diagrams as groups of discrete components communicating with each other via distinct data signal connections, it will be understood by those skilled in the art that the illustrated embodiments may be provided by a combination of hardware and software components, with some components being implemented by a given function or operation of a hardware or software system, and many of the data paths illustrated being implemented by data communication within a computer application or operating system. The structure illustrated is thus provided for efficiency of teaching the described embodiment.

The embodiments described above are intended to be exemplary only. The scope of the invention is therefore intended to be limited solely by the appended claims.

1. A method for delivering a gas containing carbon dioxide to a patient, said method comprising:
measuring a physiological parameter of breathing stability in said patient;
determining an optimal gas delivery parameter based on said physiological parameter of breathing stability; and
delivering said gas to said patient in accordance with said optimal gas delivery parameter.

2. The method as claimed in claim 1, wherein said gas containing carbon dioxide is a mixture of gases including carbon dioxide.

3. The method as claimed in claim 1, further comprising repeating the step of measuring the physiological parameter of breathing stability in the patient, after said delivering said gas, to determine an effect of said delivering on said physiological parameter.

4. The method as claimed in claim 3, further comprising repeating said steps of determining said optimal gas delivery parameter and delivering said gas to adjust said delivering consequently to said effect.

5. The method as claimed in claim 1, wherein the optimal gas delivery parameter is selected from the group consisting of a fraction of carbon dioxide in the gas and a flow rate of the gas during said delivering.

6. The method as claimed in claim 1, further comprising issuing an alarm if the physiological parameter is measured to be outside of a predetermined threshold.

7. The method as claimed in claim 1, wherein the physiological parameter is the breathing pattern for the patient, the breathing pattern including at least the respiratory amplitude.

8. The method as claimed in claim 7, wherein the physiological parameter is analyzed to obtain a breathing pattern index for the patient and the determining the gas delivery parameter is carried out using the breathing pattern index.

9. The method as claimed in claim 8, wherein the physiological parameter further includes at least one parameter selected from the group consisting of arterial hemoglobin oxygen saturation, respiratory rate, respiratory amplitude, chest movement pattern, end tidal CO2 (ETCO2) level, Rapid Eye Movement (REM) pattern, rate of apnea, rate of hypopnea, rate of desaturation, respiratory rate variability, heart rate variability, heart rate synchrony and snoring noise level.

10. A system for delivering a gas containing carbon dioxide to a patient, said system comprising:
a physiological sensor for measuring a physiological parameter of breathing stability in said patient;
a controller receiving said physiological parameter from said physiological sensor for determining an optimal gas delivery parameter based on said physiological parameter of breathing stability; and
a gas delivery sub-system having a gas source and a gas delivery controller for delivering said gas to said patient in accordance with said optimal gas delivery parameter received from said controller.

11. The system as claimed in claim 10, wherein said gas containing carbon dioxide is a mixture of gases including carbon dioxide.

12. The system as claimed in claim 10, wherein the optimal gas delivery parameter is selected from the group consisting of a fraction of carbon dioxide in the gas and a flow rate of the gas during said delivering and wherein said gas delivery controller uses said gas delivery parameter to deliver said gas from said source.

13. The system as claimed in claim 10, further comprising an alarm sub-system including an alarm emitter and an alarm controller, the alarm controller having a predetermined threshold, the alarm controller receiving the physiological parameter from the controller and controlling the alarm emitter to issue an alarm if the physiological parameter is measured to be outside of the predetermined threshold.

14. The system as claimed in claim 10, wherein the physiological parameter is the breathing pattern for the patient, the breathing pattern including at least the respiratory amplitude.

15. The system as claimed in claim 14, further comprising a breathing pattern index calculator for analyzing the physiological parameter to obtain a breathing pattern index for the patient and wherein said controller uses the breathing pattern index to determine the gas delivery parameter.

16. The system as claimed in claim 15, wherein the physiological parameter further includes at least one parameter selected from the group consisting of arterial hemoglobin oxygen saturation, respiratory rate, respiratory amplitude, chest movement pattern, end tidal CO2 (ETCO2) level, Rapid Eye Movement (REM) pattern, rate of apnea, rate of hypopnea, rate of desaturation, respiratory rate variability, heart rate variability, heart rate synchrony and snoring noise level.

17. The system as claimed in claim 10, further comprising an analysis module for analyzing said measured physiological parameter and determined gas delivery parameter to detect a trend for said patient.
